(19) United States
(12) Patent Application Publication
(10) Pub. No.: US 2023/0166109 A1
(43) Pub. Date: Jun. 1, 2023

(54) SYSTEM, METHOD AND COMPUTER PROGRAM PRODUCT FOR ENHANCED LEARNING USING BRAIN-GUIDED NON-INVASIVE BRAIN STIMULATION

(71) Applicant: Vanderbilt University, Nashville, TN (US)

(72) Inventors: Katherine Aboud, Nashville, TN (US); Laurie Cutting, Nashville, TN (US)

(21) Appl. No.: 17/993,218

(22) Filed: Nov. 23, 2022

Related U.S. Application Data
(60) Provisional application No. 63/283,786, filed on Nov. 29, 2021.

(51) Int. Cl. A61N 1/36; A61N 1/372

(52) U.S. Cl. CPC A61N 1/36092; A61N 1/36139; A61N 1/37217

(57) ABSTRACT

Described herein are methods, systems and computer program products for individualized, non-invasive brain stimulation for enhanced learning.

### CROSS REFERENCE TO RELATED APPLICATIONS

This application claims priority to and benefit of U.S. provisional patent application Ser. No. 63/283,786 filed Nov. 29, 2021, which is fully incorporated by reference and made a part hereof.

### GOVERNMENT SUPPORT CLAUSE

This invention was made with government support under Grant No. P50 HD103537 awarded by the National Institutes of Health. The government has certain rights in the invention.

### BACKGROUND

Currently, brain stimulation approaches are largely non-individualized, despite that fact that there are large, dynamic differences in how peoples' brains process information. Individualized approaches to date have used single brain imaging modalities (e.g. magnetic resonance imaging (MRI) alone) to generate targets or other parameters. Furthermore, individualized approaches are difficult to employ in clinical settings for non-scientist use.

Therefore, what are needed are systems, methods and computer program products that overcome challenges in the art, some of which are described above. Specifically, what is desired is a system, method and computer program product that provides an accessible platform for non-scientists to get brain signatures of a task, and translate them into stimulation application parameters to increase or suppress neuronal activation in order to enhance cognition. Brain imaging approaches that increase the spatial and temporal resolution additionally allow for individualized, dynamic brain stimulation, informed by brain data at a preferred resolution of milliseconds and millimeters. This can be applied in any clinical group or cognitive domain that justifies non-invasive brain stimulation. This can also specifically be applied in environments that don't have direct scientific support.

### SUMMARY

Described and disclosed herein are embodiments of systems, methods and computer program products for individualized, non-invasive brain stimulation for enhanced learning.

It should be understood that the above-described subject matter may also be implemented as a computer-controlled apparatus, a computing system, or an article of manufacture, such as a computer program product stored on a non-transient computer-readable storage medium.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF THE DRAWINGS

The accompanying drawings, which are incorporated in and constitute a part of this specification, illustrate embodiments and together with the description, serve to explain the principles of the methods and systems. The patent or application file contains at least one drawing executed in color. Copies of this patent or patent application publication with color drawing(s) will be provided by the Office upon request and payment of the necessary fee:

FIG. 1 illustrates an example of fused MRI/EEG analysis, implemented to track brain networks over the milliseconds after a word is displayed;

FIGS. 2A and 2B illustrate an example of how fused MRI/EEG can pinpoint where and when information processing can break down during reading, resulting in lower comprehension;

FIG. 3 is an example flowchart illustrating steps performed in performing individualized, non-invasive brain stimulation for enhanced learning;

FIG. 4 is a block diagram of an example computing device upon which embodiments of the invention may be implemented;

FIG. 5A illustrates example stimulus for the 2×2 congruency design, in which word pairs embedded in a sentence were incongruent (first column; incongruent words; IW) or congruent (second column; congruent words; CW), and sentence pairs were implausible (top row; orange boxes; incongruent sentences; IS) or plausible (bottom row; blue boxes; congruent sentences; CS). The current study examines CS versus IS;

FIG. 5B illustrates grand average ERP time courses from centroparietal electrodes for incongruent (orange line; IS) and congruent (blue line; CS) sentences show expected N400 and P600 effects in the one second after the sentence-final world (significant at p<0.05; and

FIG. 6 illustrates timing parameters for the sentence stimuli. Each stimulus screen was presented for 500 ms, with 100 ms between screens. After the sentence was finished, subjects saw a plus sign for 1000 ms, then a prompt of “No/Yes” for 1250 ms, during which time they had to press a button for whether the sentence did or did not make sense.

### DETAILED DESCRIPTION

Unless defined otherwise, all technical and scientific terms used herein have the same meaning as commonly understood by one of ordinary skill in the art. Methods and materials similar or equivalent to those described herein can be used in the practice or testing of the present disclosure.

As used in the specification and the appended claims, the singular forms “a,” “an,” “the,” and “data” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal embodiment. “Such as” is not used in a restrictive sense, but for explanatory purposes.

Furthermore, as used herein, the terms “word” or “words” includes a complete word, only a portion of a word, or a word segment.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

The present methods and systems may be understood more readily by reference to the following detailed description of preferred embodiments and the Examples included therein and to the Figures and their previous and following description.

Disclosed herein is a system, method and computer-program product that converts an individual's high-resolution brain patterns into an individualized stimulation protocol. Generally, a user of the disclosed a system, method and computer-program product is naïve to neuroimaging processing, and only needs to input a subject identifier to get individualized stimulation parameters for optimal stimulation gains.

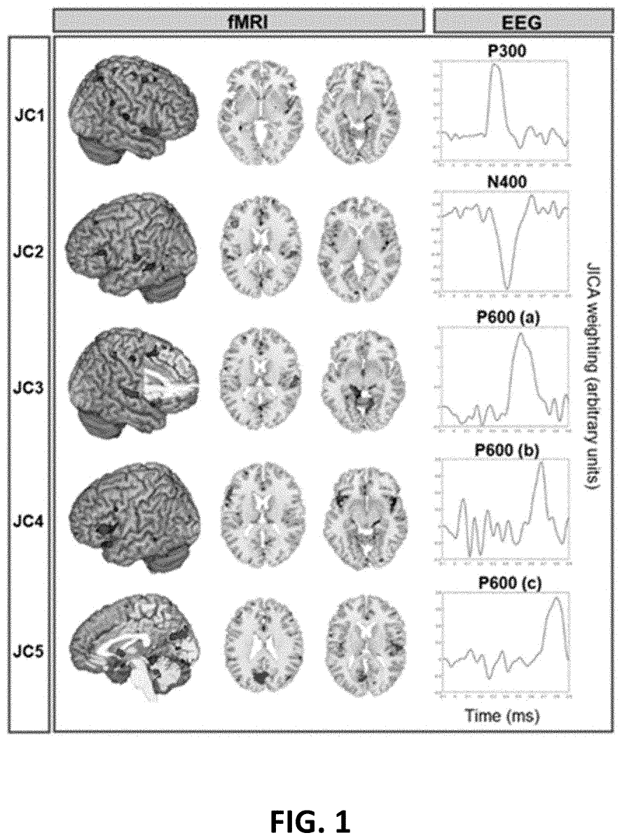

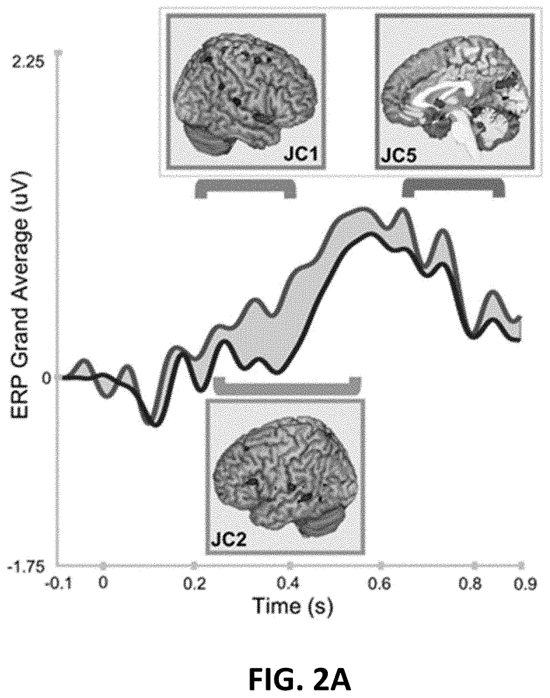

The disclosed system, method and computer-program product may be configured to automatically pull a subject's functional brain imaging data (e.g. from one or more of magnetic resonance imaging (MRI), electroencephalogram (EEG), functional near-infrared spectroscopy (fNIRS), magnetoencephalography (MEG), or other devices or combinations of devices that measure brain activation) as they view a standardized learning paradigm, along with the subject's behavioral learning patterns (e.g. what they recall from the paradigm). The disclosed system, method and computer-program product then applies automated preprocessing and modelling algorithms in individual or combined brain imaging modalities (in Matlab, for instance using SPM and EEGLab packages). One non-limiting example of this would be the use of joint independent component analysis (jICA) of EEG/MRI data to identify where and when learning happens while an individual is reading new medical facts for a test. In a current use-case, individuals study material for the Army's Expert Field Medical Badge Test while they are in the MRI and while they are wearing the EEG. An example is provided below of using use joint independent component analysis of fMRI and EEG to track the rapid (millisecond) interactions of widespread cortical networks necessary for semantic cognition beyond the single word, and identify which networks and network interactions are most critical for language comprehension (LC) ability. This example is further described in Katherine S. Aboud, Tin Q. Nguyen, Stephanie N. Del Tufo, Catie Chang, David H. Zald, Alexandra P. Key, Gavin R. Price, Bennett A. Landman and Laurie E. Cutting; Journal of Neuroscience 16 Nov. 2022, JN-RM-0529-21; DOI: https://doi.org/10.1523/JNEUROSCI.0529-21.2022, which is fully incorporated by reference and made a part hereof. jICA co-estimates MRI and EEG components. In so doing, it identifies detailed MRI spatial maps and corresponding time courses, leading to an understanding of where and when brain processes of learning occur in that individual, for instance where and what parts of the brain are most active or most suppressed when a person learns a new medical fact. FIG. 1 illustrates an example of fused MRI/EEG analysis, implemented to track brain networks over the milliseconds after a word is displayed. The peak of the EEG waveform indicates when the peak of the network activation occurs. Here, it can be seen that after a word is displayed, temporal brain regions activate ˜300 milliseconds after the word (Joint Component 1), followed by fronto-temporal language areas at ˜400 milliseconds (Joint Component 2), proceeding across other cortical networks until finally arriving at higher order comprehension areas at ˜800 ms (Joint Component 5). This degree of spatial and temporal resolution cannot be gained through other imaging modalities/analyses. The disclosed system, method and computer-program product allows for both single- and multi-modality identification of learning in the brain, depending on available technology of the user. The disclosed system, method and computer-program product then identifies which fused MRI/EEG output is most predictive of the subject's successful online learning of information. FIGS. 2A and 2B illustrate an example of how fused MRI/EEG can pinpoint where and when information processing can break down during reading, resulting in lower comprehension. If multiple brain areas are identified as important to learning, filtering algorithms are used to identify the top areas that contribute to learning (e.g. quantifying the brain areas based on their global connectivity during task, using measures of the brain regions' contribution to the spatial map, or using a priori information that points to the most important areas in the spatial map for the task, among other approaches). FIG. 2A illustrates a comparison of single subject grand averages of event-related potential (ERP) data for two representative subjects with high (red) and low (blue) LC scores. The group spatial components and related time windows (indicated by box length) for P300 (light blue), N400 (orange), and P600c (green) are overlaid onto the subject ERP responses, demonstrating the dynamic relationship between the significant components, and how those dynamics contribute to LC ability (the P300, N400, and P600). FIG. 2B illustrates mediation analysis revealed that default mode network (DMN) comprehension areas activated ˜750 ms post-stimulus (JC5; green box) are partially mediated the relationship between early semantic-phonological areas activated at ˜300 ms post-stimulus (JC1; blue box) and LC ability. The disclosed system, method and computer-program product then identifies the non-invasive brain stimulation electrode array that most closely targets the brain areas identified from the fused MRI/EEG analysis. Stimulation parameters include intensity, frequency and/or duration. Intensity ranges from ˜1-4 mA in total across multiple electrodes. Frequency can be individualized within a pre-determined frequency band depending on task (e.g. 5-7 Hz for memory paradigms). Stimulation sends current through the scalp from an anode to a cathode to either facilitate or inhibit neuronal activity. Some stimulation approaches, such as tDCS, directly act on the neurons' action potentials. Stimulation can also be used to elicit groups of neurons to fire together at a certain frequency (using tACS), which impacts communication across brain areas. In some instances, the stimulation may vary over time, while in others the stimulation is consistent over the time applied. In some instances, this may be done by interfacing the disclosed system, method and computer-program product with existing software such as, for example, HD-Targets and HD-Explore by Soterix Medical. To identify the best fit electrode array, the disclosed system, method and computer-program product contribute an algorithm to HD-Targets/HD-Explore that iteratively tests electric field maps (e.g. the simulated impact of stimulation from an electrode array) to find the array with the closest spatial match to the jICA targets. The user consequently gets an electrode array after inputting the subject identifier. The disclosed system, method and computer-program product also provides a subject's specific peak frequency value within each frequency band (from EEG), which will be used as another parameter in non-invasive brain stimulation. For all processes, cloud computing may be used to allow for rapid outputs.

The disclosed system, method and computer-program product comprise a pipeline that can be applied to many brain domains, including motor learning, attention, visual processing, etc. In some instances, the disclosed system, method and computer-program product include time-variant targets for non-invasive brain stimulation. jICA allows for detailed information into the online dynamics of brain activations related to a task. Currently, the majority of non-invasive brain stimulation techniques consistently target the same brain areas over the course of a simulation session, despite the fact that brain activations dynamically and flexibly change during tasks in a way that is specific to an individual. The disclosed system, method and computer-program product can, in some instances, be used to input time-variant stimulation protocols.

In some instances, the disclosed system, method and computer-program product can be deployed to whole classrooms. Once a person's EEG-MRI alignment is established, classroom applications can employ real-time adjustments to non-invasive brain stimulation based on the person's online EEG patterns alone. For instance, it can be identified that EEG pattern X is related to MRI pattern Y, while EEG pattern A is related to MRI pattern B. If it is desired to enhance areas in Y when they occur, but suppress areas in B when they occur, then online EEG measurements of X and A (from the subject's previous MRI-EEG mapping) can be used to make online stimulation target adjustments.

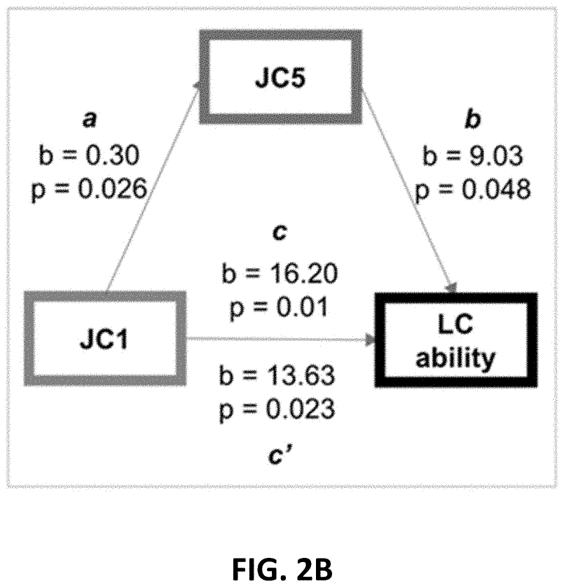

FIG. 3 illustrates an exemplary flowchart for performing a method of individualized, non-invasive brain stimulation for enhanced learning. At 302, brain data of a person is obtained. In some instances, brain data are obtained while the person is performing a learning activity. For example, the learning activity may be a specific learning activity such as medical learning, marksmanship, and the like.

Generally, though not required, the brain data comprise high-resolution brain data (defined as data with spatial and temporal information at a preferred resolution on the millisecond and millimeter scale). For example, the high resolution brain imaging comprises magnetic resonance imaging (MRI) and/or electroencephalogram (EEG) data.

At 304, one or more areas of a brain of the person are identified for stimulation based on the brain data. In instances where the brain data comprise MRI and/or EEG data, identifying the one or more areas of the brain of the person for stimulation comprises performing joint independent component analysis (jICA) of the MRI and EEG data. The jICA analysis identifies one or more detailed MRI spatial maps and corresponding time courses, leading to a high resolution understanding of brain processes. At least one of the one or more detailed MRI spatial maps and corresponding time courses is identified as most predictive of the subject's successful online learning of information. The identified one detailed MRI spatial map and corresponding time course is masked to contain only one or more greatest contributing brain areas (based either on weighting value of the area, or a priori characterization of the region). A non-invasive brain stimulation electrode array that most closely targets the one or more greatest contributing brain areas is identified from the fused MRI/EEG analysis. For example, identifying the non-invasive brain stimulation electrode array that most closely targets the one or more greatest contributing brain areas identified from the fused MRI/EEG analysis may be done by using HD-Targets and HD-Explore by Soterix Medical. Using HD-Targets and HD-Explore by Soterix Medical to identify a best fit electrode array generally comprises contributing an algorithm to HD-Targets/HD-Explore that iteratively tests electric field maps (e.g. the simulated impact of stimulation from an electrode array) to find the electrode array with the closest spatial match to the jICA targets.

At 306, the identified one or more areas of the brain are stimulated using the identified electrode array wherein the stimulation results in enhanced learning. In some instances, the person's specific peak frequency value within each frequency band (from EEG) may be used as another parameter in the non-invasive brain stimulation. Furthermore, in some instances, the stimulation for stimulating the identified one or more areas of the brain varies over time. Such variance may occur in the form of varying peak voltage, frequencies, duration, and the like.

It is to be appreciated that the above described steps can be performed by computer-readable instructions executed by a processor. As used herein, a processor is a physical, tangible device used to execute computer-readable instructions.

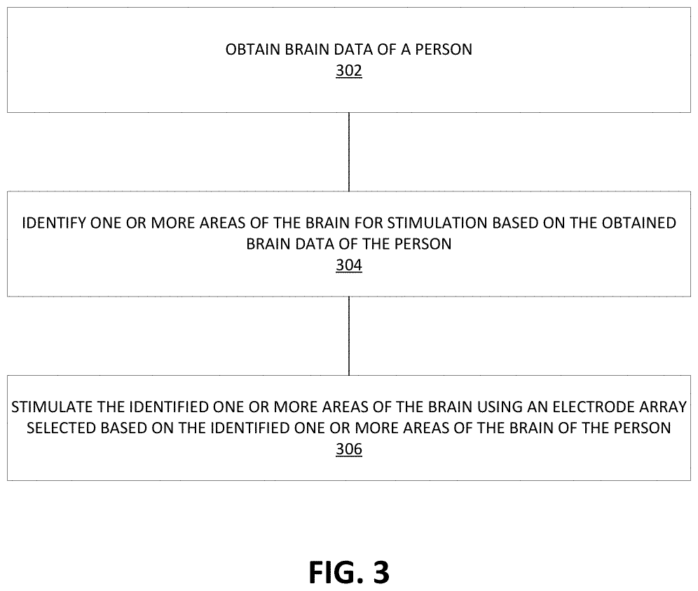

When the logical operations described herein are implemented in software, the process may execute on any type of computing architecture or platform. For example, referring to FIG. 4, an example computing device upon which embodiments of the invention may be implemented is illustrated. In particular, at least one processing device described above may be a computing device, such as computing device 1000 shown in FIG. 4. For example, computing device 1000 may be a component of the cloud computing and storage system. Computing device 1000 may comprise all or a portion of server. The computing device 1000 may include a bus or other communication mechanism for communicating information among various components of the computing device 1000. In its most basic configuration, computing device 1000 typically includes at least one processing unit 1006 and system memory 1004. Depending on the exact configuration and type of computing device, system memory 1004 may be volatile (such as random access memory (RAM)), non-volatile (such as read-only memory (ROM), flash memory, etc.), or some combination of the two. This most basic configuration is illustrated in FIG. 4 by dashed line 1002. The processing unit 1006 may be a standard programmable processor that performs arithmetic and logic operations necessary for operation of the computing device 1000.

Computing device 1000 may have additional features/functionality. For example, computing device 1000 may include additional storage such as removable storage 1008 and non-removable storage 1010 including, but not limited to, magnetic or optical disks or tapes. Computing device 1000 may also contain network connection(s) 1016 that allow the device to communicate with other devices. Computing device 1000 may also have input device(s) 1014 such as a keyboard, mouse, touch screen, scanner, etc. Output device(s) 1012 such as a display, speakers, printer, etc. may also be included. The additional devices may be connected to the bus in order to facilitate communication of data among the components of the computing device 1000. All these devices are well known in the art and need not be discussed at length here. Though not shown in FIG. 4, in some instances computing device 1000 includes an interface. The interface may include one or more components configured to transmit and receive data via a communication network, such as the Internet, Ethernet, a local area network, a wide-area network, a workstation peer-to-peer network, a direct link network, a wireless network, or any other suitable communication platform. For example, interface may include one or more modulators, demodulators, multiplexers, demultiplexers, network communication devices, wireless devices, antennas, modems, and any other type of device configured to enable data communication via a communication network. Interface may also allow the computing device to connect with and communicate with an input or an output peripheral device such as a scanner, printer, and the like.

The processing unit 1006 may be configured to execute program code encoded in tangible, computer-readable media. Computer-readable media refers to any media that is capable of providing data that causes the computing device 1000 (i.e., a machine) to operate in a particular fashion. Various computer-readable media may be utilized to provide instructions to the processing unit 1006 for execution. Common forms of computer-readable media include, for example, magnetic media, optical media, physical media, memory chips or cartridges, a carrier wave, or any other medium from which a computer can read. Example computer-readable media may include, but is not limited to, volatile media, non-volatile media and transmission media. Volatile and non-volatile media may be implemented in any method or technology for storage of information such as computer readable instructions, data structures, program modules or other data and common forms are discussed in detail below. Transmission media may include coaxial cables, copper wires and/or fiber optic cables, as well as acoustic or light waves, such as those generated during radio-wave and infra-red data communication. Example tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

In an example implementation, the processing unit 1006 may execute program code stored in the system memory 1004. For example, the bus may carry data to the system memory 1004, from which the processing unit 1006 receives and executes instructions. The data received by the system memory 1004 may optionally be stored on the removable storage 1008 or the non-removable storage 1010 before or after execution by the processing unit 1006.

Computing device 1000 typically includes a variety of computer-readable media. Computer-readable media can be any available media that can be accessed by device 1000 and includes both volatile and non-volatile media, removable and non-removable media. Computer storage media include volatile and non-volatile, and removable and non-removable media implemented in any method or technology for storage of information such as computer readable instructions, data structures, program modules or other data. System memory 1004, removable storage 1008, and non-removable storage 1010 are all examples of computer storage media. Computer storage media include, but are not limited to, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by computing device 1000. Any such computer storage media may be part of computing device 1000.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-ROMs, hard drives, or any other machine-readable storage medium wherein, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high level procedural or object-oriented programming language to communicate with a computer system. However, the program(s) can be implemented in assembly or machine language, if desired. In any case, the language may be a compiled or interpreted language and it may be combined with hardware implementations.

### Example of Characterizing Semantic Cognition

Language comprehension requires the rapid retrieval and integration of contextually-appropriate concepts (“semantic cognition”). Current neurobiological models of semantic cognition are limited by the spatial and temporal restrictions of single modality neuroimaging and lesion approaches. This is a major impediment given the rapid sequence of processing steps that have to be coordinated to accurately comprehend language. Through the use of fused functional magnetic resonance imaging and electroencephalography analysis in humans (n=26 adults; 15 female), we elucidate a temporally and spatially specific neurobiological model for real-time semantic cognition. We find that semantic cognition in the context of language comprehension is supported by trade-offs between widespread neural networks over the course of milliseconds. Incorporation of spatial and temporal characteristics, as well as behavioral measures, provide convergent evidence for the following progression: a hippocampal-anterior temporal phonological-semantic retrieval network (peaking at ˜300 ms after the sentence final word); a fronto-temporal thematic semantic network (−400 ms); a hippocampal memory update network (−500 ms); an inferior frontal semantic-syntactic re-appraisal network (−600 ms); and nodes of the default mode network associated with conceptual coherence (−750 ms). Additionally, in typical adults, mediatory relationships between these networks are significantly predictive of language comprehension ability. These findings provide a conceptual and methodological framework for the examination of speech and language disorders, with additional implications for the characterization of cognitive processes and clinical populations in other cognitive domains.

The present study identifies a real-time neurobiological model of the meaning processes required during language comprehension (i.e. “semantic cognition”). Using an application of fused magnetic resonance imaging and electroencephalography in humans, we found that semantic cognition during language comprehension is supported by a rapid progression of widespread neural networks related to meaning, meaning integration, memory, re-appraisal, and conceptual cohesion. Relationships between these systems were predictive of individuals' language comprehension efficiency. Our findings use fused neuroimaging analysis to elucidate language processes. In so doing, this study provides a new conceptual and methodological framework in which to characterize language processes and guide the treatment of speech and language deficits/disorders.

The ability to rapidly extract meaningful information from language is a fundamental human skill needed to navigate the social world. Adequate language comprehension (LC) ability in the context of complex language (i.e. beyond single word processing) requires the engagement of multiple brain networks responsible for accessing and combining appropriate concepts (i.e. “semantic cognition”). The breakdown of this complex process is a key clinical marker across a range of neurological disorders (Mueller, Hermann, Mecollari, & Turkstra, 2018; O'Sullivan, Brownsett, & Copland, 2019; Smith & Caplan, 2018), but data-driven neurobiological characterization of LC and LC ability is limited. Evidence from functional magnetic resonance imaging (fMRI) and electroencephalography (EEG) studies identify spatial and temporal patterns of semantic cognition, respectively. Theories that span both of these modalities agree to some extent that meaning processes stem from rapid interactions between word retrieval processes in the temporal lobe and combinatorial meaning processes in the frontal lobe (Friederici & Gierhan, 2013; P Hagoort, 2013). However, despite numerous studies of complex language processes, beyond single words, the “interplay [of these networks] in the service of language understanding remains to be specified” (Friederici, 2015). Underspecification of these processes is due in large part to the spatial and temporal limitations of single modality neuroimaging that prevent a real-time elucidation of brain networks that support LC (Dronkers, Ivanova, & Baldo, 2017; Lee Osterhout, Kim, & Kuperberg, 2012). This is a major impediment given the rapid sequence of processing steps that have to be coordinated to accurately comprehend language. Here we use joint independent component analysis (V D Calhoun, Adali, Pearlson, & Kiehl, 2006) of fMRI and EEG to track the rapid (millisecond) interactions of widespread cortical networks necessary for semantic cognition beyond the single word, and identify which networks and network interactions are most critical for LC ability.

Previous fMRI and lesion studies have highlighted a number of frontal, temporal, and parietal brain regions that support semantic cognition (i.e. where semantic cognition occurs), though the specific nature of each region's contribution is still debated (Friederici, 2011; Friederici & Gierhan, 2013; Peter Hagoort, 2013; Jefferies, 2013). Brain studies point to a semantic memory network centered in temporal structures, such as the middle temporal gyms (MTG) and anterior temporal lobes (ATL), which supports the retrieval of word meaning from long-term memory (Peter Hagoort, 2013; Jefferies, 2013). However, both regions have also been implicated in context-dependent meaning access (i.e. semantic control) (Davey et al., 2016; Ferstl, Neumann, Bogler, & von Cramon, 2008; Whitney, Kirk, O'Sullivan, Lambon Ralph, & Jefferies, 2012), and it is unclear whether the MTG or ATL is the primary retrieval hub. Lesion and neuroimaging work has suggested that the MTG may act as a connection point between the ATL-centered retrieval network and fronto-temporal semantic control network, primarily comprising the MTG and the inferior frontal gyms (IFG). The IFG is also consistently linked to a more general role in combinatorial processes that unify individual words into a multi-word context (Davey et al., 2016; Friederici, 2011; Peter Hagoort, 2005). Hagoort et al. (2014) suggest that the left IFG contains a gradation of functionality related to the binding of information, with ventral IFG supporting semantic combinatorial processes, and dorsal IFG supporting syntactic processes. At the highest level of comprehension, studies have found a consistent set of domain-general processing areas in the default mode network (DMN) whose inter-regional correlations are associated with sentence-and story-level information (Baldassano et al., 2017a; Ferstl et al., 2008; March, 2011; Simony et al., 2016), with a specific role for the posterior midline (precuneus/posterior cingulate; PCU/PCC) in building the coherence of ideas (Ferstl et al., 2008; Whitney et al., 2009). fMRI studies consequently reveal that adequate semantic cognition requires interactions between temporal and frontal language areas that support the access and integration of meaning. However, the limited temporal resolution in fMRI prevent characterization of when and how these networks interact.

In a largely separate body of literature, event-related potential (ERP) studies measure rapid synchronized firing of neuronal ensembles that occur after a stimulus of interest (i.e. when semantic cognition occurs). Peak amplitudes of the corresponding waveforms vary based on semantic cognition demands. This task sensitivity allows insight into the underlying cognitive functions of ERP signals. To date, ERP work has demonstrated that meaning access and integration occurs ˜300-800 ms after word onset (Kutas & Federmeier, 2011). ERP studies have focused on characterizing cognition related to waveforms within this time window in order to develop a model of sub-processes that result in adequate semantic cognition. This includes studies on the canonical N400 effect, a negative waveform whose amplitude reflects the difficulty of meaning access (Kutas & Federmeier, 2011). Recent attention has also been given to two positive waveforms, the P300 and P600, implicated in semantic memory and combinatorial processes, respectively, but their role in semantic cognition is heavily debated (Brouwer, Fitz, & Hoeks, 2012; DeLong, Quante, & Kutas, 2014; Kuperberg, 2007; L Osterhout, 1997; Riby & Orme, 2013). While ERP work provides a critical temporal framework in which to examine semantic cognition, limitations in source localization have prevented more complete, data-driven spatio-temporal models of semantic cognition, particularly in the context of sentence comprehension (Friederici, 2015).

The respective temporal and spatial limitations of fMRI and EEG have prevented a comprehensive, data-driven model of semantic cognition, and consequently, restricted our understanding of language-related disorders. In the present study, we address the single-modality limitations of fMRI and ERP through a novel application of fused fMRI and ERP analysis, called joint independent component analysis (jICA), to track both where and when semantic cognition occurs during sentence comprehension, and via this approach, identify the neural progression that underpins typical language comprehension ability (V D Calhoun et al., 2006; Mijović et al., 2012). JICA takes advantage of cross-subject variability to simultaneously estimate independent components from subjects' fMRI spatial maps and ERP timecourses. The output are joint components that include a spatial map and corresponding timecourse, and subject-specific loadings that indicate how similar a subject's brain signatures are to the component. This approach allows us to identify network activation changes on the millisecond time scale (Mijović et al., 2012). Here, we identify (1.) the progression of rapid brain network exchanges needed to extract meaning from language, and (2.) which of these brain signals contribute to LC ability.

Thirty right-handed male and female adult participants were recruited from the community. All participants were native English speakers with normal or corrected-to-normal vision, no history of major psychiatric illness, and no contraindication to magnetic resonance imaging (MRI). To ensure that subjects had IQ within the normal range and did not have dyslexia, we administered the Kaufman Brief Intelligence Test—matrices subtest (Bain & Jaspers, 2010) and Woodcock Johnson Reading Mastery Test (WRMT) III—Letter Word Identification (LWID) and Word Attack (WA) subtests (McGrew, Schrank, & Woodcock, 2007). Behavioral metrics confirmed that subjects had IQ within the normal range (minimum>85 ss; mean=111.52 +/−8.48) and basic reading ability (minimum>85 ss; mean=105.94 +/−7.9). Out of the original subject pool, n=4 were excluded due to motion artifacts (n=3) and inability to complete the two sessions (n=1). The final analysis included 26 adults (mean age=25.36 +/−3.69; 15 female). Participants gave written informed consent at the beginning of the study, with procedures carried out in accordance with Vanderbilt University's Institutional Review Board (IRB). Participants received compensation for behavioral and neuroimaging testing as per the study's IRB

In order to assess subject language comprehension (LC) ability, we administered the WRMT-III Passage Comprehension subtest. The WRMT-III requires subjects to read a sentence and fill in a missing word. To determine basic reading (BR) ability, scores on the WRMT-III Letter Word Identification (LWID) and Word Attack (WA) subtests were averaged and converted to z-scores. To parse the individual contributions of P600 sub-components, we additionally collected behavioral metrics on vocabulary, syntactic, and conceptual integration ability. Receptive vocabulary was measured using the Peabody Picture Vocabulary Test (PPVT) (Dunn & Dunn, 2007); syntactic ability was measured using the Woodcock Johnson IV (WJ-IV)—Sentence Fluency sub-test (Schrank, Mather, & McGrew, 2014). Working memory was assessed using the Weschler Adult Intelligence Scale IV (WAIS-IV) (Drozdick, Wahlstrom, Zhu, & Weiss, 2012), backward and sequential digit span subtests. Conceptual integration was measured using an in-house paradigm based on previous work (Graves, Binder, Desai, Conant, & Seidenberg, 2010) in which subjects viewed a grid of four pictures, and were instructed to combine two of the pictures to create a compound word (e.g. a picture of a house and a picture of a tree would be identified as a “tree-house”). Only two of the pictures could form a compound word, with the other two pictures acting as distractors. Distractor pictures were target pictures from other trials in the test. N=23 subjects completed all behavioral tests, and regression analyses were run in this subset (see Statistical Approach).

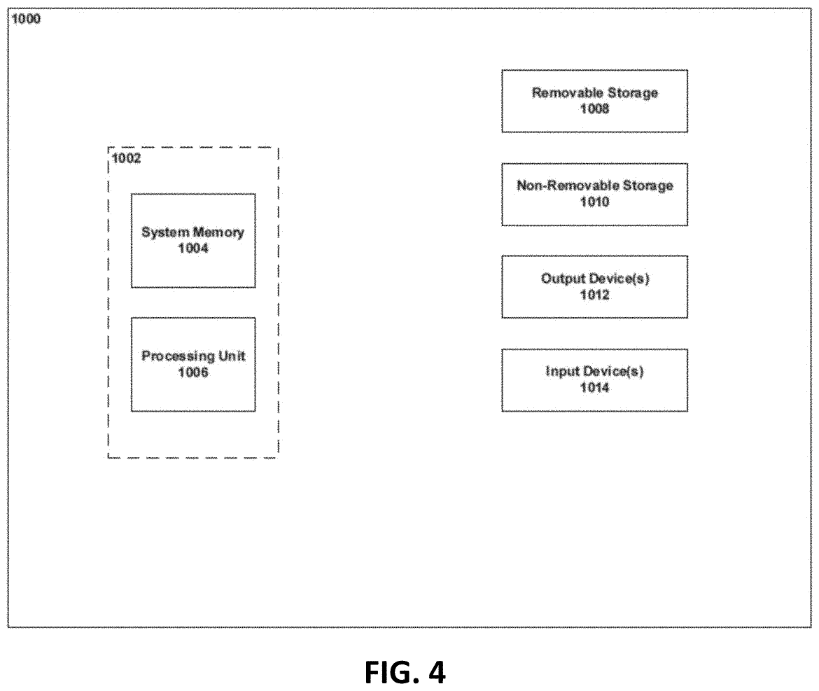

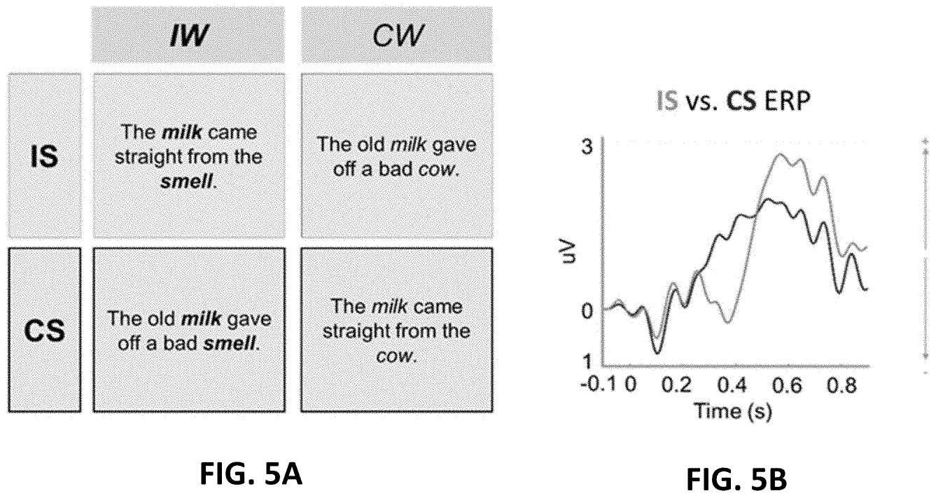

Stimuli were created to capture both word- and sentence-level congruence effects, but only sentence-congruence effects are within the scope of the present study. Specifically, we employed a novel 2×2 sentence reading design that manipulated lexical congruency (i.e. whether or not embedded word pairs were semantically related to one another) and sentence congruence (i.e. whether or not the sentence “made sense”; see FIGS. 5A and 5B). Stimuli were constructed in sets of two sentence frames (4-11 words). Though not examined in the present study, the sentence-final critical word was either primed by a preceding word (>0.07 association strength; South Florida Association Norms (Nelson, McEvoy, & Schreiber, 2004)), or not primed (<0.07 association strength), and either made the sentence congruent or incongruent in meaning (see FIGS. 5A and 5B). All critical words were included in both congruent and incongruent sentence conditions. Incongruent sentences either contained information contradictory to known world properties (e.g. The bird spread its fingers) or to world experience (e.g. Amy got in trouble for getting dirt on her mud), or contained internally contradictory information (e.g. The glasses did not work and made her eyes see). A task-probe on whether the sentence made sense (see below) revealed that subjects were able to distinguish incongruent sentences from congruent sentences with a high degree of accuracy (>90%). The paradigm resulted in four conditions: 1.) Congruent word pairs, congruent sentence (CWCS), 2.) Incongruent word pairs, congruent sentence (IWCS), 3.) Congruent word pairs, incongruent sentence (CWIS), and 3.) Incongruent word pairs, incongruent sentence (IWIS). Word congruency effects were insignificant and outside of the scope of the present paper. In the analytical pipeline (described below) sentence congruency effects were determined by comparing congruent sentence conditions (CWCS and IWCS) to incongruent sentence conditions (CWIS and IWIS). The methodological approach ensured that congruent and incongruent sentences were syntactically identical, with only the final word differing across the comparison.

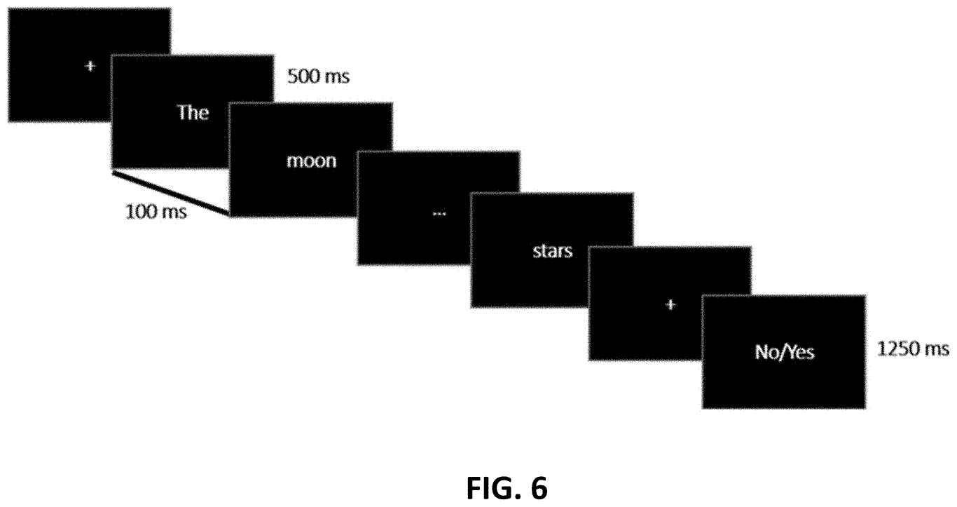

Two separate lists of stimuli were constructed. Each list contained a total of 192 sentences (48 sentences/condition) presented across 4 runs (duration=˜6 minutes/run). In order to minimize repetition effects related to repeated sentence frames, sentence presentation order was randomized within the list. Lists were counterbalanced across fMRI and EEG per subject. FMRI and EEG administration order was counterbalanced across subjects (see below). During each session, sentences were presented one word at a time (see FIG. 6). Words had white letters centered on a black background, Comic Sans MS font, size 32, at a visual angle of 25 degrees. Each word was presented for 500 ms, with a 100 ms pause between words. To ensure task attention and sentence comprehension, subjects were probed after the end of each sentence about whether the sentence did or did not make sense (i.e. sentence congruency measure). The sentence's terminal word was followed by a 1000 ms break (indicated by a plus sign), then a probe of “yes/no”, during which the subject had 1250 ms to respond on a button box. All subjects had high accuracy (>90%) for the sentence probe, confirming that incongruent sentences were highly identifiable, and that subjects stayed on task. Due to high performance on the probe, all sentences were included in the final analysis.

In order to counteract any learning effects related to the task, the fMRI and EEG sessions were separated by an average of 6.1 +/−3.5 months (range: 3 days-1.19 years), and fMRI/EEG administration order was counterbalanced across subjects (n=13 subjects performed the EEG session first). Subjects were additionally counterbalanced on which of the two stimuli lists they received for their first session, as well as response hand.

All fMRI scans were acquired at Vanderbilt University Institute of Imaging Sciences on one of two Philips Achieva 3T MR scanners with a 32-channel head coil. Functional images were acquired using a gradient echo planar imaging sequence with 40 (3 mm thick) slices with no gap and consisted of 4 runs (single run duration=6 minutes; 160 dynamics per run). Slices were collected parallel to the anterior-posterior commissure plane. Additional imaging parameters for functional images included echo time (TE)=30 msec, FOV 240×240×120 mm, 75 degree flip angle, and repetition time (TR)=2200 msec, and 3 mm3 voxels. Image processing was completed using Matlab R2018b and SPM12 (Friston et al., 1994). We utilized an event-related design, with the events timed to the sentence-final critical word. Preprocessing included slice timing correction (corrected to central slice, n=20), realignment of volumes to the mean functional image, coregistration of the T1 to the MNI template, segmentation-based normalization of functional images to a standardized space, smoothing (kernel of 8 mm3) and motion correction using ART. Subjects with>20% motion outliers (defined with a z-threshold of 9) were excluded from the analysis (n=1). For each subject, contrast maps of the sentence final word were generated per condition (CWCS, IWCS, CWIS, IWIS) versus an explicitly modeled plus-sign baseline. These subject-level contrasts were input into the joint ICA pipeline.

All EEG data were acquired at the Vanderbilt Kennedy Center, using a 128 channel geodesic sensor net (EGI, Inc., Eugene, Oreg.). Data were sampled at 250 Hz with filters set to 0.1-30 Hz. The vertex was used as the reference during data acquisition. Data processing was completed using NetStation and Matlab. EEG data were segmented into epochs of 1000 ms, starting 100 ms before the onset of the critical word. For all conditions, the critical word was in the sentence-final position. Recordings were re-referenced to an average reference. Ocular and muscle artifacts were identified through automated and manual artifact identification processes; contaminated electrodes in each trial were rejected and trials with greater than ten rejected electrodes were excluded from analysis. To be included in the statistical analysis, individual condition ERPs were based on a minimum of 20 trials; n=2 subjects were excluded due to excessive motion artifacts. Confirmatory analysis of the waveforms revealed expected N400 effects across sentence conditions. The N400 was defined as the mean voltages in a 300-600 ms latency window when compared to the 100 ms prestimulus baseline, pulled from centroparietal electrodes (Electrodes: 54, 55, 62, 80, 81, 32, 7, 107 (Kutas & Federmeier, 2011)). Pre-processed time signals for each condition were averaged across subjects, then entered into a grand average across subjects per condition. These grand averages for each of the 4 conditions were input into the joint ICA pipeline (i.e. the same conditions as the fMRI jICA inputs). Difference waves were generated for Incongruent—Congruent Words, and Incongruent—Congruent Sentences, and the maximum negative peak within the N400 time window (300-600 ms) per subject was input into a one-sample t-test. The N400 effect was significant for word congruence (t(25)=−4.19; p<0.001; d=−1.68) and sentence congruence (t(25)=−12.12; p<0.001; d=−4.85) manipulations. These findings confirm that our paradigm captured expected EEG patterns (see FIG. 5B).

Fusion analysis was performed using the Fusion ICA Toolbox (FIT) in Matlab, and followed processing protocols established by Calhoun et al. (2006) and Mijović et al. (2012) (see also (Edwards, Calhoun, & Kiehl, 2012; Ouyang et al., 2015)), which were developed for parallel fMRI/EEG acquisition (notably, parallel acquisition has been found to be more ideal for this approach than simultaneous acquisition; V D Calhoun et al., 2006; Mijović et al., 2012). In jICA, independent components for fMRI and EEG are simultaneously estimated. Compared to other multimodal analysis approaches, jICA allows for the spatial and temporal components of EEG and fMRI, respectively, to influence each other, and is consequently considered to truly be a “fused” data analysis approach (Mijović et al., 2012). In jICA, the spatial fMRI maps and the ERP component timecourse are concatenated into a subject×data input matrix (the ERP timecourse is upsampled using a cubic spline interpolation so that it is the same dimensionality as the spatial fMRI vector (Mijović et al., 2012)). The fMRI and ERP data are first-level contrast map and grand average timecourse (averaged across centroparietal electrodes), respectively, for one condition. Consequently, the only within-subject data in the pipeline is condition. The model assumes that ERP peaks and BOLD responses change in a similar way across subjects. This approach provides robust, high-quality data decompositions (Mijović et al., 2012) which have been validated across a number of cognitive substrates and populations (Vince D. Calhoun et al., 2006; Vince D Calhoun & Adali, 2009; Edwards et al., 2012; Ouyang et al., 2015). The jICA algorithm outputs group-level, joint independent components that includes information for each modality (i.e. one component includes both an ERP time course and a spatial map). Condition-specific maps and time-courses are back-reconstructed to allow identification of how each condition contributes to the cross-condition components. The strength of this contribution is reflected by a subject- and condition-specific scalar parameter loading (i.e. a measure of how “strong” the component signal is within that subject and condition), which can be used to statistically identify condition differences per component. This means that each subject had 4 scalar loadings (1 per condition) that could be included in statistical models. A limitation of this ICA stacking method is that it assumes each condition has a similar underlying signal that only differs in magnitude. However, in the present study, a measurement of spatial divergence (Renyi divergence) revealed that the average divergence across conditions was very low (divergence<2). As voxel-by-voxel statistical tests were not run in the spatial maps, multiple comparison correction was not employed. For display, the spatial maps are normalized and voxels with a value of z>2.6 were displayed, which identifies the voxels that are the highest contributors to the component (i.e. >2.6 standard deviations above the mean contribution, equivalent to p<.005). This is consistent with procedures of ICA in MRI (V D Calhoun et al., 2006; Vince D Calhoun, Liu, & Adali, 2009; Edwards et al., 2012; Himberg, Hyvärinen, & Esposito, 2004; Mijović et al., 2012).

The Infomax algorithm was used to identify joint components. To determine the ideal number of components, we followed protocols established by Artoni et al. (2014) and Himberg et al. (2004)(Artoni et al., 2014; Himberg et al., 2004). First, we used ICASSO to identify the number of stable components. ICASSO iteratively runs ICA to determine the stability of generated components. As recommended by Himberg et al. (2004), we set the component number, k, to the subject number (k=26), and performed 50 ICASSO iterations. Final component number was determined using the stability index (Iq), which reflect the internal stability of a component. K=15 components was the smallest k value to meet a high Iq threshold (mean Iq>0.95; minimum Iq>0.90)(James et al., 2014; Turner et al., 2012; Himberg et al., 2004). Qualitative follow-up examination revealed that adjacent component amounts (e.g. k=14 and k=16) resulted in nearly identical findings, while examinations of 1.) low total components (e.g. k=4) showed a merging of significant subcomponents (e.g. the P600 was a single component, whereas higher component amounts subdivided the P600 into sub-components that significantly contributed to the raw data), and 2.) high total components (e.g. k=21) showed a division of relevant subcomponents so that they did not meet contribution thresholds.

As done in Calhoun et al. (2006), we took advantage of the ERP signals to identify components that reflected noise versus true brain signals. We applied the following criteria: 1) Components had to contribute >1 standard deviation (SD) of variance to the grand mean of the EEG signal (n=5 components removed) (Edwards et al., 2012); 2) we used the findpeaks Matlab function to identify any components with an excessive number of peaks (outliers defined as >30 peaks; n=1 component removed); 3) after non-noisy components were identified, the remaining components were screened to identify positive or negative temporal waveforms that fell within the expected time window of semantic cognition (300-800 ms post-stimuli) (Hoedemaker & Gordon, 2017; Mollo, Jefferies, Cornelissen, & Gennari, 2018; Pulvermüller, 2012). This screening resulted in five spatiotemporal components that shared characteristics with the P300 (n=1 component), N400 (n=1 component), and P600 (n=3 components; referred to in the present paper as P600a, P600b, and P600c, in order of peak latency). To determine replicability of the results, we performed a split-halves validation analysis (Vabalas et al., 2019). Subjects were randomized into two subgroups of 13 subjects each (with 4 conditions per subject). Separate JICA analyses, identical to the full group analyses, were run for each group. We used Matlab's findpeaks to identify components with peaks within the range of each joint component's peak from the full analysis, and resulting time courses were manually reviewed. This resulted in 5 JCs per subgroup that showed temporal characteristics of the P300, N400, P600a, P600b, and P600c. To determine whether spatial maps were replicated, first, MNI labels were generated from the full analysis at a reduced z-threshold (z>1.5). Subgroup MNI labels were then compared to the full analysis labels. For each label and subgroup, the subgroup received a value of 1 if it had activation in the full analysis region, and 0 if it did not (at z>1.5). This resulted in binary vectors for each subgroup, which were then compared to one another using intraclass correlation coefficient (ICC). The subgroup JICA spatial maps showed fair (0.4-0.6) to good (>0.6) ICC values with one another (Brandt et al., 2013) with the exception of JC3 (see below). Whole-brain conjunction maps across subgroups (z>1.5) for all components (JCs 1-5) also revealed overlap in the key language areas seen in the full analysis. Here we provide the ICC values for each spatial comparison (all significant at p<0.005), r-values for each temporal comparison (all significant at p<0.001), and whole-brain overlap in regions of interest within the language and comprehension network across subgroups: 1.) JC1: ICC=0.64, r=0.40 (regions: bilateral ATL; mPFC; left SMG); 2.) JC2: ICC=0.47, r=0.91 (regions: left MTG, left ATL); 3.) JC3: ICC=0.30, r=0.83 (regions: bilateral parahippocampal gyrus/hippocampus, STG); 4.) JC4: ICC=0.63, r=0.38 (regions: left ventral IFG, left ATL); and 5.) JC5: ICC=0.58, r=0.64 (regions: bilateral precuneus (PCU)).

Once peaks were identified, confirmatory one-sided t-tests were run to ensure that the joint component loadings showed expected significant effects related to semantic cognition demands (incongruent vs. congruent sentences; one-sided; all p-values Benjamini-Hochberg FDR corrected for five tests). In order to characterize behavioral correlates of the joint components, two ANCOVA's per component were run to ascertain the relationship between the JC loadings. The first model per component included language-related metrics (basic reading, vocabulary, syntax, and conceptual integration, controlling for condition). The second model per component included working memory metrics (digit span backwards and sequential, controlling for condition). The dependent variable was the component loading per condition across subjects, and the independent variables were the behavioral metrics, which allowed for control of covariance across behavioral measures and allowed us to specifically isolate independent behavioral predictors of the component loading. Conditions were treated as repeated measures (i.e. there were 4 component loading values per subject), and as there were no significant interactions across analyses with condition, condition was included as a control variable. These regressions revealed significant associations between the JC's and behavior. All p-values across variables in the ten tests were corrected together using Benjamini-Hochberg FDR correction.

To ascertain which joint components were predictive of language comprehension ability (LC; as defined by standard scores from the WRMT-III passage comprehension subtest), five multiple regression analyses were run (one per component) with LC ability as the dependent variable, and the joint component loadings as the independent variable (with condition as a control). All p-values were corrected using Benjamini-Hochberg FDR correction for five tests. Condition did not show any significant interactions with the component loadings in predicting LC ability and so the condition interaction was removed from the models. As recommended by Luck (2005), separate analyses were run per component to see which of the independent components predicted LC ability. Three components were significantly predictive of LC ability (JC1, JC2 and JC5). Lastly, mediation analysis was run to determine whether, within the set of components significantly related to LC ability (JC1, JC2 and JC5), the relationship between early components and LC ability is mediated by later components (i.e. whether effects of early components on LC ability “go through” later components). To test this, we used the Mediation Toolbox (Wager, 2022) to run pairwise mediation analyses in the order of the temporal sequence, with the mediator the later components in the time series (as recommended by mediation time series analysis in Agler & De Boek, 2017; results FDR-corrected). This resulted in three mediation analyses: 1.) JC1 and LC ability, mediated by JC2; 2.) JC1 and LC ability, mediated by JC5; and 3.) JC2 and LC ability mediated by JC5. All results were bootstrapped in the Mediation Toolbox with 10,000 iterations, and resulting p-values were corrected using Benjamini-Hochberg FDR correction for three tests.

To provide additional evidence for the role of each JC, we used Neurosynth (www.neurosynth.org) to identify cognitive terms that are significantly associated with the primary nodes of each JC. Neurosynth is a meta-analytic data-based with over 14,000 studies, which can estimate the key terms most closely associated with a particular coordinate (search radius of 6 mm). We input peak coordinates for the five largest clusters of each JC into the Neurosynth localization software. Neurosynth performs an association test which runs an ANOVA to test whether a region is more consistently present in studies that mention a key term vs. studies that do not mention a key term. After excluding the following term categories: brain regions, clinical populations, clinical diagnoses, methods (e.g. “stimulation”), generic, non-cognitive terminology (e.g. “task”) and close repeats of key words (i.e. “semantic” and “semantics”), we selected the top 3 key terms for each of the peak coordinates. In addition to providing a z-score for term-to-seed match, Neurosynth also provides an r-value for the correlation between the meta-analytic functional connectivity map of a specific term (e.g. “semantic memory”) and the meta-analytic resting state connectivity of the seed area (e.g. connectivity to the left MTG). The r-score is another view of how the seed's network corresponds with the term's associated network, and we included the term with the top r-value for each seed region in a table.

Adult participants (n=26) read sentences during fMRI and, in a separate session, while EEG data were collected. Sentences were constructed to vary in semantic cognition demands, with the sentence-final word determining whether the sentence was congruent or incongruent in overall meaning (i.e. whether the sentence did or did not make sense; see FIGS. 5A and 5B). Sentence frames were identical across congruent and incongruent conditions, thus comparisons of incongruent vs. congruent conditions allowed for isolation of semantic rather than syntactic processing (see FIG. 5A). After each sentence, participants indicated whether the sentence made sense or not via button press.

Subject-level fMRI spatial maps and ERP time-courses (averaged across trials) were input into the joint independent component analysis (jICA) pipeline. JICA allows for identification of spatial maps and corresponding time courses related to a stimulus, in this case the final word of the sentence. As done in previous studies, we used the time course of the ERP waveforms as the orienting framework for the joint components so that the peak of the joint component ERP time courses indicates the temporal order of the corresponding spatial activations (Mijović et al., 2012). We then examined which joint components corresponded with subjects' written language comprehension ability.

The jICA approach revealed five spatiotemporal components within the canonical time window of semantic cognition (300-800 ms after the critical word; see FIG. 1). Temporally, these signals reflected characteristics of the P300 (n=1 joint component), N400 (n=1), and P600 (n=3). After FDR correction for multiple comparisons (Benjamini-Hochberg FDR correction for 5 comparisons), results revealed significant sentence congruence effects (congruent vs. incongruent) for each of the five joint components, which we include here as confirmation of appropriate component identification: JC1: semantic retrieval network and the P300 (t(25)=5.93; p-corr=<0.001; d=2.37); JC2: semantic control network and the N400 (t(25)=−6.96; p-corr<0.001; d=−2.78); memory network and P600a (t(25)=−1.75; p-corr=0.046; d=−0.7); semantic-syntactic network and P600b (t(25)=−2.64; p-corr=0.011; d=−1.06); JC5: comprehension network and P600c (t(25)=−2.27; p-corr=0.02; d=−0.91). In order to ascertain the distinct functions of each sub-peak, we ran two ANCOVA's per component to identify which subject behaviors of (1.) word reading, vocabulary, syntax, and conceptual integration, and 2.) working memory ability best predicted jICA loadings on each component (see Methods; all p-values Benjamini-Hochberg FDR corrected for all p-values within the ten tests).

### Joint Component 1 (JC1)—Semantic Memory

JC1 localized to the hippocampus, bilateral/right ATL (BA 22/38), MTG, medial prefrontal cortex (mPFC), left supramarginal gyms (SMG), areas in the dorsal attention network (frontal eye fields, inferior parietal lobule), and the anterior cingulate cortex (ACC). The component had a positive peak that occurred at ˜300 ms post-stimulus onset, consistent with the P300 component. An ANCOVA revealed that JC1 was significantly positively associated with basic reading ability (t(86)=3.96, p-corr=0.002; d=0.85) and working memory (t(88)=3.72, p-corr=0.003; d=0.79; digit span sequential).

### Joint Component 2 (JC2)—Semantic Control

The second joint component localized to canonical language areas, including bilateral temporal regions (superior temporal gyms (STG), MTG, and ATL), bilateral frontal areas (IFG and dorsolateral prefrontal cortex), cingulate gyms, PCU, and the motor/cerebellar regions. This component temporally had a negative peak at ˜400 ms post-stimulus, which fell into the N400 effect time-window for our average ERP results (see FIG. 5B). An ANCOVA revealed that JC2 was significantly negatively associated with syntax (t(86)=−4.20, p-corr=0.001; d=−0.91; sentence fluency) and working memory (t(88)=−3.64, p-corr=0.003; d=−0.78; digit span sequential).

### Joint Component 3 (JC3)—Memory Schema

The first P600 component corresponded with co-activation of language and memory regions, including bilateral STG (BA 22), parahippocampal areas, right (SMG), ACC, PCU, premotor and sensory areas, insula, and the cerebellum. This component had a positive peak latency at ˜500 ms post-stimulus, which fell into the early P600 effects window in the average ERP findings (see FIG. 5B). The temporal component will henceforth be referred to as P600a. An ANCOVA revealed that JC3 was significantly negatively associated with vocabulary ability (t(86)=−2.62, p-corr=0.034; d=−0.57) and working memory (t(88)=−3.03, p-corr=0.012; d=−0.65; digit span backwards). Syntactic ability was also associated with the JC3; however, syntactic ability did not predict JC3 loadings alone, and inclusion in the model decreased the model's R2, revealing it as a suppressor variable.

### Joint Component 4 (JC4)—Semantic-Syntactic Re-appraisal

The second joint component within the P600 range (P600b) corresponded with co-activated clusters in bilateral ventral and left dorsal IFG, as well as activation in bilateral STG, parahippocampal and fusiform gyri, PCC, sensory areas and the cerebellum. This component exhibited classic spatiotemporal properties of the “syntactic” P600, with a positive peak latency at ˜600 ms post-stimulus. To ascertain the specificity of this component to semantic processes, we ran an ANCOVA to examine the relationship of this component to semantic and syntactic ability. JC4 had significant positive associations with both vocabulary ability (t(86)=2.60, p-corr=0.034; d=0.56) and syntax (t(86)=3.08; p-corr=0.011; d=0.66; sentence fluency), each contributing independent variance, with a trending relationship with working memory (t(88)=2.23, p-corr=0.076; d=0.47; digit span sequential).

### Joint Component 5 (JC5)—Conceptual Coherence

The final joint component within the P600 range (P600c) had co-activation of regions within the default mode network (DMN), particularly in the PCU, but with smaller loadings in the mPFC, left AG, and PCC. Additional co-activations could also be seen in bilateral STG, sensory areas, the insula, and the cerebellum. This component had a late positive peak latency at ˜750 ms post-stimulus. An ANCOVA revealed that JC5 was significantly, positively predicted by conceptual integration (t(86)=3.36, p-corr=0.005; d=0.72).

Spatio-Temporal Patterns of LC ability

In the second set of analyses, we aimed to identify how individual differences in LC ability are related to the spatiotemporal signals described above. We found that global LC ability (defined by standard scores from the WRMT-III passage comprehension subtest, controlling for condition) was positively related to JC1 (P300; bilateral ATL; t(89)=3.13; p-corr=0.006; d=0.66); negatively related to JC2 (N400; left IFG and left MTG; t(89)=−3.11; p-corr=0.006; d=−0.66) , and positively related to JC5 (P600c; DMN; t(89)=2.58; p-corr=0.019; ; d=0.55; Benjamini-Hochberg FDR correction for 5 comparisons). This suggests that during sentence comprehension, efficient LC is associated with greater reliance on earlier automated retrieval processes centered in the ATL (BA 22/38) and hippocampus, and late comprehension processes in the DMN, with decreased reliance on activation within the extended semantic control network in the N400 time window (see FIG. 2A). We were next interested in whether the effect of earlier components on LC ability would be mediated by (i.e. “go through”) later components. Specifically, we anticipated that the effect of JC1 on LC ability would be mediated by JC2 and JC5 and the effect of JC2 on LC ability would be mediated by JC5. We found all three analyses had trending or significant mediations (all p-values Benjamini-Hochberg FDR corrected for three comparisons). JC2 had a trending mediation effect on JC1's relationship with LC ability (t(89)=1.52; b=5.10; d=0.32; p-corr=.077), and JC5 had a significant partial mediation on JC1 and LC ability (t(89)=1.55; b=2.57; d=0.33; p-corr=0.040). Additionally, JC5 had a trending mediation effect on JC2's relationship with LC ability (t(89)=−1.69; b=−2.51; d=−0.36; p-corr=0.073; see FIG. 2B). This provides initial evidence that in addition to the impact of individual components on LC ability, there may be dependencies between brain networks that support LC ability, including a potential neural path between early semantic-phonological brain areas to later comprehension areas.

We applied a novel fused MRI/EEG approach to elucidate language processes and identify a temporally and spatially specified neurobiological model for LC ability. Our combined results suggest that semantic cognition during language processing involves a system of rapid sequential engagements of multiple cortical networks. Specifically, semantic cognition during sentence reading is first marked by (1) early signals centered in the hippocampus and bilateral ATL (peak at ˜300 ms; corresponding with P300 component), closely followed by and overlapping with (2) ATL coupling with the broader fronto-temporal language network (peak at ˜400 ms; corresponding with N400 component). These early language network patterns, respectively mapping to regions associated with semantic retrieval and control (Davey et al., 2015; Whitney, Kirk, O'Sullivan, Lambon Ralph, & Jefferies, 2011), are followed by three networks that fall within the P600 waveform and have distinct spatial, temporal, and behavioral characteristics: (3) a hippocampal and right STG network corresponding with sequential working memory ability (peak at ˜500 ms), (4) a ventral and dorsal IFG network corresponding with vocabulary and syntactic ability (peak at ˜600 ms), and lastly, (5) the DMN (particularly the posterior midline) corresponding with conceptual integration ability (peak at ˜750 ms). The particular mappings for each component reflect and extend previous psycholinguistic theories of sentence comprehension (Fengler, Meyer, & Friederici, 2016; Friederici, 2002).

To better contextualize the potential functional contributions of each joint component, we utilized the meta-analytic platform Neurosynth (www.neurosynth.org), which provides information on the functions most associated with a given brain region. Although this approach relies on reverse inference rather than experimental manipulation, it provides a data-driven framework for interpreting the possible functions associated with the temporal flow of information observed in the present study.

Our earliest components, JC1 and JC2, map to phonological/semantic memory and semantic integration areas, respectively. Interestingly, they also have overlapping time windows with peaks at ˜300 ms and ˜400 ms for JC1 and JC2, respectively (i.e. they are independent, but co-occur). For JC1 (P300), the mappings to the hippocampus, temporal lobes, and mPFC areas are consistent with previously described memory circuits, specifically automated spreading activations during the retrieval of dominant semantic characteristics of an item (Davey et al., 2016). These areas are coactivated with frontal and temporal regions associated in Neurosynth with phonological processing. Covariate analyses revealed that JC1 is positively related to basic word reading processes (i.e. sounding out words) and working memory. The combined findings suggest that JC1 may support phonological-semantic binding processes related to word reading. The sensitivity of JC1 to sentence congruence (with greater loadings for congruent sentences) suggests that these word-level processes are assisted by prior semantic context; i.e. JC1 interacts with predictive processing prior to the sentence-final word. This interpretation is consistent with broader theories of the P300 (Polich, 2007; Azizian et al., 2006). Integrative theories suggest that P300 sources support template-matching processes in which information that matches an internal representation (e.g. automated semantic memory processes facilitated by sentences that are congruent with a person's real-world experience) results in a larger P300 amplitude than non-matched information. Our findings suggest that these early activations in frontal and temporal areas work in parallel with language processing areas in JC2 (N400). JC2's regions fall within the well-described semantic control network; i.e. regions that constrain word meaning retrieval based on the thematic content of the previous text (also called thematic semantic processes)(Davey et al., 2015, 2016; Thompson et al., 2018; Whitney et al., 2012). The negative relationship between JC2 and sentence fluency ability, working memory ability, and sentence congruence suggests that JC2 is more prevalent with increased difficulty, whether at the subject or stimulus level. This interpretation is consistent with theories suggesting that the N400 supports probabilistic, thematic interpretation of a word based on the preceding context, and traces to a wave of activation across fronto-temporal language areas (Kutas & Federmeier, 2011). Combined, JC1 and JC2 appear to form a complex of memory and meaning processes that overlap in the left superior ATL. Of note, neither JC1 nor JC2 correlated with vocabulary ability, despite being sensitive to semantic congruence manipulations, and mapping to canonical semantic processing areas. It is possible that JC1 and JC2's functionalities are less related to the depth of vocabulary knowledge than to the predictive and contextual retrieval of semantic information. However, further study is needed to distinguish the precise form of semantic cognition related to these components.

The JC1/JC2 complex is followed by three components that all fall within the P600 time window, but which have distinct etymologies. JC3 is the earliest peak in the P600 time window, peaking at ˜500 ms post-stimulus, and its spatial map includes the hippocampus, prefrontal and posterior midline areas related to recognition memory, working memory, and language. JC3 was also found to be significantly negatively related to an individual's working memory ability. The combination of latency, localization, and behavior in JC3 provides evidence that this component shares similarities with the characteristics of the late positive component (LPC; sometimes also referred to as the P300b). The LPC has been linked to post-hoc memory-schema updates, in which unexpected information either triggers greater long-term memory retrieval processes than expected information already stored in working memory (Olichney et al., 2000), or requires greater reliance on updates to an existing memory scheme (DeLong et al., 2014; Richter, 2019), resulting in a larger LPC amplitude. This effect is dependent on the ease of this process, and so individuals with more automated memory and language processes would be expected to have a lower jICA loadings, as is the case in our findings. In the context of psycholinguistic theory, the joint component described here may reflect communication between semantic and syntactic binding areas (STG)(Frankland & Greene, 2015; Skeide, Brauer, & Friederici, 2014) and memory schema encoding areas (Milivojevic, Varadinov, Vicente Grabovetsky, Collin, & Doeller, 2016), in order to support “structured semantic combinations” that reference previously stored structures (Frankland & Greene, 2015).

JC4 (P600b) shares spatial and temporal features with the canonical P600 effect, with a peak at ˜600 ms post-stimuli and spatial map centered on the left IFG. The P600 effect has been proposed to reflect syntactic-only re-appraisal (Osterhout & Holcomb, 1992), semantic-syntactic re-appraisal (Kuperberg, 2007), semantic integration (Brouwer et al., 2012), and/or domain-general processes (Burkhardt, 2007; Shen, Fiori-Duharcourt, & Isel, 2016), among others. In the present study, our findings support the theory that the P600b effect is driven by both semantic and syntactic re-appraisal processes during sentence comprehension. First, JC4 includes well-known semantic and syntactic frontal areas (ventral and dorsal IFG, respectively)(Hagoort, 2005), as well having significant and independent positive associations with vocabulary and syntactic ability. Additionally, our paradigm argues against a syntax-only interpretation: the sentence frames in which only the final word determined congruency ensure that syntactic processes for congruent versus noncongruent sentences are identical, and consequently allows for isolation of semantic processes. Our findings support previous suggestions that reassessment in the context of sentence comprehension (in typical adults) is a dynamic process that does involve the semantic system at ˜600 ms post-stimulus (Kuperberg, 2007).

The last component, JC5 (P600c), has a spatial map that includes key nodes of the default mode network that peaks at ˜750 ms post-stimulus. This component is also significantly associated with a subject's conceptual integration ability (see Methods). The location, latency, and behavior of JC5 is consistent with a role in situation model processing, i.e. the updating of new information into the reader's internal representation of the text (Aboud, Bailey, Petrill, & Cutting, 2016; Baldassano et al., 2017b; Whitney et al., 2009). Specifically, the primary loading in the Cuneus/PCU directly overlaps with several studies on narrative processing, which have found that the PCU is specifically sensitive to event boundaries within stories (Baldassano et al., 2017b; Whitney et al., 2009). Whitney et al. (2009) has proposed that this portion of the PCU acts to update situation models at key narrative moments (i.e. “narrative shifts”), which corresponds with the region's more general association with episodic memory (Zhang & Li, 2012). Previous work has also shown that incongruent information within a story elicits stronger responses in the PCU. Recent ERP work found strong evidence for a relationship between a P600 at similar latency and situation model updating (Burkhardt, 2007). Our finding that JC5 is sensitive to incongruent sentences is the first to consolidate fMRI and ERP literature findings to provide joint evidence that situation model updating may occur at the tail-end of the P600 effect in sentence processing.

Overall, our results provide evidence that semantic processing of sentences involves early phonological-semantic and thematic semantic processes occurring in the hippocampus/ATL/SMG and fronto-temporal language circuit, respectively. These early activations are followed by hippocampal-based schematic memory processes potentially related to semantic-syntactic schema in the STG (Frankland & Greene, 2015; Skeide et al., 2014); inferior frontal semantic-syntactic re-appraisal; and lastly, conceptual integration in the DMN. From this high-resolution examination of semantic cognition, we were next able to provide evidence for a neurobiological model of typical LC ability. Through regression and mediation analyses, we found that less efficient LC is marked by early decreased reliance on a focal network for phonological-semantic memory network (JC1); this is followed by greater reliance on a wider language network related to the N400 (JC2), potentially via the left ATL hub shared by JC1 and JC2; then this complex is proceeded by a decreased reliance in the comprehension areas of the DMN (JC5). Mediation analysis showed that the relationship between early phonological-semantic areas and LC ability is mediated by DMN comprehension areas, with trending support for a progression of mediation effects in which the impact of early semantic-phonological signals on LC ability is mediated, or “goes through”, thematic semantic and comprehension networks. This suggests that the relationship across components, as well as the individual components themselves, are important in predicting LC ability. Because our methods do not provide evidence for directionality, future studies with causal designs should test the dependent relationship across components, including whether early semantic/phonological components drive comprehension networks that lead to LC ability, and/or whether later DMN comprehension areas gate the impact that early components have on LC ability. Additional studies should also test whether joint components centered on different electrode groupings and captured at different time points in a sentence are able to identify networks that predict or interact with the ones described in the current manuscript. It is unclear from our findings why JC3 and JC4 are not related to LC ability. It is possible that the memory and linguistic binding processes in JC3 and JC4 are more relevant to global reading success in longer texts, or that these signals would interact with LC ability in different populations with atypical LC ability. Additional study is needed to ascertain in which contexts these components may interact with LC ability.

The present study demonstrates the feasibility of applying a fused MRI/EEG approach to disentangle the complex temporal unfolding of processes necessary for LC. However, the data-driven approach in the present study has a key limitation: because of the absence of experimental manipulations in the stimulation paradigm, it was not possible to specifically test the neurocognitive profiles for each joint component. Nevertheless, the current results lay the groundwork for future studies employing the joint ICA pipeline to test a range of language paradigms and clinical populations. Future studies should examine how interventions such as non-invasive brain stimulation may impact these signals so that causal relationships between components and LC ability/outcomes can be established.

By fusing MRI and EEG to elucidate language processes and identify a temporally and spatially specified neurobiological model for LC ability, this approach provides a new conceptual and methodological paradigm in which to examine speech and language deficits/disorders, and may have additional implications for the examination and treatment of clinical populations in other cognitive domains.

### REFERENCES

Unless otherwise noted, each of the below references are fully incorporated by reference and made a part hereof:

Aboud, K. S., Bailey, S. K., Petrill, S. A., & Cutting, L. E. (2016). Comprehending text versus reading words in young readers with varying reading ability: Distinct patterns of functional connectivity from common processing hubs. Developmental Science, 1-25. https://doi.org/10.1111/desc.12422

Ager, R. & De Boeck, P (2017). On the Interpretation and Use of Mediation: Multiple Perspectives on Mediation Analysis. Front. Psych. https://doi.org/10.3389/fpsyg.2017.01984

Artoni, F., Menicucci, D., Delorme, A., Makeig, S., & Micera, S. (2014). Full Length Articles RELICA: A method for estimating the reliability of 3 independent components 4Q1.https://doi.org/10.1016/j.neuroimage.2014.09.010

Azizian, A., Freitas, A. L., Watson, T. D., & Squires, N. K. (2005). Electrophysiological correlates of categorization: P300 amplitude as index of target similarity. https://doi.org/10.1016/j.biopsycho.2005.05.002

Bain, S. K., & Jaspers, K. E. (2010). Test Review: Review of Kaufman Brief Intelligence Test, Second Edition: Kaufman, A. S., & Kaufman, N. L. (2004). Kaufman Brief Intelligence Test, Second Edition. Bloomington, Minn.: Pearson, Inc. Journal of Psychoeducational Assessment, 28(2), 167-174. https://doi.org/10.1177/0734282909348217

Baldassano, C., Chen, J., Zadbood, A., Pillow, J. W., Hasson, U., & Norman, K. A. (2017a). Discovering Event Structure in Continuous Narrative Perception and Memory. Neuron, 95(3), 709-721.e5. https://doi.org/10.1016/j.neuron.2017.06.041

Baldassano, C., Chen, J., Zadbood, A., Pillow, J. W., Hasson, U., & Norman, K. A. (2017b). Discovering Event Structure in Continuous Narrative Perception and Memory. https://doi.org/10.1016/j.neuron.2017.06.041

Brouwer, H., Fitz, H., & Hoeks, J. (2012). Getting real about Semantic Illusions: Rethinking the functional role of the P600 in language comprehension. Brain Research, 1446, 127-143. https://doi.org/10.1016/j.brainres.2012.01.055

Burkhardt, P. (2007). The P600 reflects cost of new information in discourse memory. NeuroReport, 18(17), 1851-1854. https://doi.org/10.1097/WNR.0b013e3282f1a999

Calhoun, V D, Adali, T., Pearlson, G. D., & Kiehl, K. A. (2006). Neuronal chronometry of target detection: fusion of hemodynamic and event-related potential data. Neurolmage, 30(2), 544-553. https://doi.org/10.1016/j.neuroimage.2005.08.060

Calhoun, Vince D., Adali, T., Kiehl, K. A., Astur, R., Pekar, J. J., & Pearlson, G. D. (2006). A method for multitask fMRI data fusion applied to schizophrenia. Human Brain Mapping, 27(7), 598-610. https://doi.org/10.1002/hbm.20204

Calhoun, Vince D, & Adali, T. (2009). Feature-based fusion of medical imaging data. IEEE Transactions on Information Technology in Biomedicine: A Publication of the IEEE Engineering in Medicine and Biology Society, 13(5), 711-720. https://doi.org/10.1109/TITB.2008.923773

Calhoun, Vince D, Liu, J., & Adali, T. (2009). A review of group ICA for fMRI data and ICA for joint inference of imaging, genetic, and ERP data. Neurolmage, 45(1 Suppl), S163-72. https://doi.org/10.1016/j.neuroimage.2008.10.057

Davey, J., Cornelissen, P. L., Thompson, H. E., Sonkusare, S., Hallam, G., Smallwood, J., & Jefferies, E. (2015). Automatic and Controlled Semantic Retrieval: TMS Reveals Distinct Contributions of Posterior Middle Temporal Gyms and Angular Gyms. The Journal of Neuroscience : The Official Journal of the Society for Neuroscience, 35(46), 15230-15239. https://doi.org/10.1523/JNEUROSCI.4705-14.2015

Davey, J., Thompson, H. E., Hallam, G., Karapanagiotidis, T., Murphy, C., De Caso, I., . . . Jefferies, E. (2016). Exploring the role of the posterior middle temporal gyms in semantic cognition: Integration of anterior temporal lobe with executive processes. Neurolmage, 137, 165-177. https://doi.org/10.1016/j.neuroimage.2016.05.051

DeLong, K. A., Quante, L., & Kutas, M. (2014). Predictability, plausibility, and two late ERP positivities during written sentence comprehension. Neuropsychologia, 61, 150-162. https://doi.org/10.1016/j.neuropsychologia.2014.06.016

Dronkers, N. F., Ivanova, M. V., & Baldo, J. V. (2017, Oct. 1). What do language disorders reveal about brain-language relationships? From classic models to network approaches. Journal of the International Neuropsychological Society, Vol. 23, pp. 741-754. https://doi.org/10.1017/S1355617717001126

Drozdick, L. W., Wahlstrom, D., Zhu, J., & Weiss, L. G. (2012). The Wechsler Adult Intelligence Scale—Fourth Edition and the Wechsler Memory Scale—Fourth Edition. In Contemporary intellectual assessment: Theories, tests, and issues, 3rd ed. (pp. 197-223). New York, N.Y., US: The Guilford Press.

Dunn, L. M., & Dunn, D. (2007). Peabody Picture Vocabulary Test (5th ed.). Pearson.

Edwards, B. G., Calhoun, V. D., & Kiehl, K. A. (2012). Joint ICA of ERP and fMRI during error-monitoring. Neurolmage, 59(2), 1896-1903. https://doi.org/10.1016/j.neuroimage.2011.08.088

Fengler, A., Meyer, L., & Friederici, A. D. (2016). How the brain attunes to sentence processing: Relating behavior, structure, and function. Neurolmage, 129, 268-278. https://doi.org/10.1016/j.neuroimage.2016.01.012

Ferstl, E. C., Neumann, J., Bogler, C., & von Cramon, D. Y. (2008). The extended language network: a meta-analysis of neuroimaging studies on text comprehension. Human Brain Mapping, 29(5), 581-593. https://doi.org/10.1002/hbm.20422

Frankland, S. M., & Greene, J. D. (2015). An architecture for encoding sentence meaning in left mid-superior temporal cortex. Proceedings of the National Academy of Sciences of the United States of America, 112(37), 11732-11737. https://doi.org/10.1073/pnas.1421236112

Friederici, A. D. (2002). Towards a neural basis of auditory sentence processing. Trends in Cognitive Sciences, 6(2), 78-84. https://doi.org/10.1016/S1364-6613(00)01839-8

Friederici, A. D. (2011). The brain basis of language processing: from structure to function. Physiological Reviews, 91(4), 1357-1392. https://doi.org/10.1152/physrev.00006.2011

Friederici, A. D. (2015). The Neuroanatomical Pathway Model of Language: Syntactic and Semantic Networks. In Neurobiology of Language. https://doi.org/10.1016/B978-0-12-407794-2.00029-8

Friederici, A. D., & Gierhan, S. M. E. (2013). The language network. Current Opinion in Neurobiology, 23(2), 250-254. https://doi.org/10.1016/j.conb.2012.10.002

Friston, K. J., Holmes, A. P., Worsley, K. J., Poline, J. -P, Frith, C. D., & Frackowiak, R. S. J. (1994). Statistical parametric maps in functional imaging: A general linear approach. Human Brain Mapping, 2(4), 189-210. https://doi.org/10.1002/hbm.460020402

Graves, W. W., Binder, J. R., Desai, R. H., Conant, L. L., & Seidenberg, M. S. (2010). Neural correlates of implicit and explicit combinatorial semantic processing. Neurolmage, 53(2), 638-646. https://doi.org/10.1016/j.neuroimage.2010.06.055

Hagoort, P. (2013). MUC (Memory, Unification, Control) and beyond. In Front Psychol (Vol. 4). https://doi.org/10.3389/fpsyg.2013.00416

Hagoort, Peter. (2005). On Broca, brain, and binding: a new framework. Trends in Cognitive Sciences, 9(9), 416-423. https://doi.org/10.1016/j.tics.2005.07.004

Hagoort, Peter. (2013). MUC (Memory, Unification, Control) and beyond. Frontiers in Psychology, 4, 416. https://doi.org/10.3389/fpsyg.2013.00416

Himberg, J., Hyvärinen, A., & Esposito, F. (2004). Validating the independent components of neuroimaging time series via clustering and visualization. Neurolmage, 22(3), 1214-1222. https://doi.org/10.1016/J.NEUROIMAGE.2004.03.027

Hoedemaker, R. S., & Gordon, P. C. (2017). The onset and time course of semantic priming during rapid recognition of visual words. Journal of Experimental Psychology: Human Perception and Performance, 43(5), 881-902. https://doi.org/10.1037/xhp0000377

James, A. G., Tripathi, S. P., & Kilts, C. D. (2014). Estimating brain network activity through back-projection of ICA components to GLM maps. Neuroscience Letters, 564, 21-26. http://dx.doi.org/10.1016/j.neulet.2014.01.056

Jefferies, E. (2013). The neural basis of semantic cognition: converging evidence from neuropsychology, neuroimaging and TMS. Cortex; a Journal Devoted to the Study of the Nervous System and Behavior, 49(3), 611-625. https://doi.org/10.1016/j.cortex.2012.10.008

Kuperberg, G. R. (2007). Neural mechanisms of language comprehension: Challenges to syntax. Brain Research, 1146, 23-49. https://doi.org/10.1016/J.BRAINRES.2006.12.063

Kutas, M., & Federmeier, K. D. (2011). Thirty years and counting: finding meaning in the N400 component of the event-related brain potential (ERP). Annual Review of Psychology, 62, 621-647. https://doi.org/10.1146/annurev.psych.093008.131123

Luck, Steven. (2005). An Introduction to The Event-Related Potential Technique.

Mar, R. A. (2011). The Neural Bases of Social Cognition and Story Comprehension. Annual Review of Psychology, 62, 103-134. https://doi.org/10.1146/annurev-psych-120709-145406

McGrew, K. S., Schrank, F. A., & Woodcock, R. W. (2007). Woodcock-Johnson III Normative Update. Rolling Meadows, Ill., United States: Riverside Publishing.

Mijović, B., Vanderperren, K., Novitskiy, N., Vanrumste, B., Stiers, P., Bergh, B. Van den, . . . Vos, M. De. (2012). The “why” and “how” of JointICA: results from a visual detection task. Neurolmage, 60(2), 1171-1185. https://doi.org/10.1016/j.neuroimage.2012.01.063

Milivojevic, B., Varadinov, M., Vicente Grabovetsky, A., Collin, S. H. P., & Doeller, C. F. (2016). Coding of Event Nodes and Narrative Context in the Hippocampus. The Journal of Neuroscience: The Official Journal of the Society for Neuroscience, 36(49), 12412-12424. https://doi.org/10.1523/JNEUROSCI.2889-15.2016

Mollo, G., Jefferies, E., Cornelissen, P., & Gennari, S. P. (2018). Context-dependent lexical ambiguity resolution: MEG evidence for the time-course of activity in left inferior frontal gyms and posterior middle temporal gyms. Brain and Language, 177-178, 23-36. https://doi.org/10.1016/j.bandl.2018.01.001

Mueller, K. D., Hermann, B., Mecollari, J., & Turkstra, L. S. (2018, Oct. 21). Connected speech and language in mild cognitive impairment and Alzheimer's disease: A review of picture description tasks. Journal of Clinical and Experimental Neuropsychology, Vol. 40, pp. 917-939. https://doi.org/10.1080/13803395.2018.1446513

Nelson, D. L., McEvoy, C. L., & Schreiber, T. A. (2004). The University of South Florida free association, rhyme, and word fragment norms. Behavior Research Methods, Instruments, and Computers, 36(3), 402-407. https://doi.org/10.3758/BF03195588

O'Sullivan, M., Brownsett, S., & Copland, D. (2019, Oct. 1). Language and language disorders: Neuroscience to clinical practice. Practical Neurology, Vol. 19, pp. 380-388. https://doi.org/10.1136/practneurol-2018-001961

Olichney, J. M., Van Petten, C., Paller, K. A., Salmon, D. P., Iragui, V. J., & Kutas, M. (2000). Word repetition in amnesia: Electrophysiological measures of impaired and spared memory. Brain, 123(9), 1948-1963. https://doi.org/10.1093/brain/123.9.1948

Osterhout, L. (1997). On the Language Specificity of the Brain Response to Syntactic Anomalies: Is the Syntactic Positive Shift a Member of the P300 Family? Brain and Language, 59, 494-522. Retrieved from https://pdfs.semanticscholar.org/4b9b/dfacb648516f8f472b49b9bd16f97534d9da.pdf

Osterhout, Lee, & Holcomb, P. J. (1992). Event-related brain potentials elicited by syntactic anomaly. Journal of Memory and Language, 31(6), 785-806. https://doi.org/10.1016/0749-596X(92)90039-Z

Osterhout, Lee, Kim, A., & Kuperberg, G. (2012). The Neurobiology of Sentence Comprehension. Cambridge Handbook of Psycholinguistics, 365-389.

Ouyang, X., Chen, K., Yao, L., Hu, B., Wu, X., Ye, Q., & Guo, X. (2015). Simultaneous changes in gray matter volume and white matter fractional anisotropy in Alzheimer's disease revealed by multimodal CCA and joint ICA. Neuroscience, 301, 553-562. https://doi.org/10.1016/j.neuroscience.2015.06.031

Polich J. Updating P300: an integrative theory of P3a and P3b. Clin Neurophysiol. 2007 October;118(10):2128-48. doi: 10.1016/j.clinph.2007.04.019. Epub 2007 Jun. 18. PMID: 17573239; PMCID: PMC2715154.

Pulvermüller, F. (2012). Meaning and the brain: The neurosemantics of referential, interactive, and combinatorial knowledge. Journal of Neurolinguistics, 25(5), 423-459. https://doi.org/10.1016/j.jneuroling.2011.03.004

Riby, L. M., & Orme, E. (2013). A familiar pattern? Semantic memory contributes to the enhancement of visuo-spatial memories. Brain and Cognition, 81(2), 215-222. https://doi.org/10.1016/J.BANDC.2012.10.011

Rice, G. E., Caswell, H., Moore, P., Hoffman, P., & Lambon Ralph, M. A. (2018). The Roles of Left Versus Right Anterior Temporal Lobes in Semantic Memory: A Neuropsychological Comparison of Postsurgical Temporal Lobe Epilepsy Patients. Cerebral Cortex, 28(4), 1487-1501. https://doi.org/10.1093/cercor/bhx362

Richter, F. (2019). Prediction errors indexed by the P3 track the updating of complex long-term memory schemas. 1-40. https://doi.org/10.1101/805887

Schrank, F. A., Mather, N., & McGrew, K. S. (2014). Woodcock-Johnson IV Test of Achievement. Rolling Meadows, Ill.: Riverside Publishing.

Selig, J. P., & Preacher, K. J. (2008, June). Monte Carlo method for assessing mediation: An

interactive tool for creating confidence intervals for indirect effects [Computer software]. Available from http://quantpsy.org/.

Shen, W., Fiori-Duharcourt, N., & Isel, F. (2016). Functional significance of the semantic P600. NeuroReport, 27(7), 548-558. https://doi.org/10.1097/WNR.0000000000000583

Simony, E., Honey, C. J., Chen, J., Lositsky, 0., Yeshurun, Y., Wiesel, A., & Hasson, U. (2016). Dynamic reconfiguration of the default mode network during narrative comprehension. Nature Communications, 7(1), 12141. https://doi.org/10.1038/ncomms12141

Skeide, M. A., Brauer, J., & Friederici, A. D. (2014). Syntax gradually segregates from semantics in the developing brain. Neurolmage, 100, 106-111. https://doi.org/10.1016/j.neuroimage.2014.05.080

Smith, K. M., & Caplan, D. N. (2018, Oct. 1). Communication impairment in Parkinson's disease: Impact of motor and cognitive symptoms on speech and language. Brain and Language, Vol. 185, pp. 38-46. https://doi.org/10.1016/j.bandl.2018.08.002

Thompson, C., Walenski, M., Europa, E., & Caplan, D. (2018). Neural networks for sentence comprehension and production: an ALE-based meta-analysis of neuroimaging studies. Frontiers in Human Neuroscience, 12. https://doi.org/10.3389/conf.fnhum.2018.228.00076

Turner, J. A., Calhoun, V. D., Michael, A., van Erp, T. G. M., Ehrlich, S., Segall, J. M., Gollub R. L., Csernansky, J., Potkin, S. G., Ho B., Bustillo, J., Schulz, S. C., & Wang L. (2012). Heritability of Multivariate Gray Matter Measures in Schizophrenia. Twin Research and Human Genetics, 15(3), 324-335. doi: 10.1017/thg.2012.1.

Vabalas A, Gowen E, Poliakoff E, Casson A J (2019) Machine learning algorithm validation with a limited sample size. PLoS ONE 14(11): e0224365. https://doi.org/10.1371/journal.pone.0224365

Wager, T (2022). MediationToolbox (https://github.com/canlab/MediationToolbox), GitHub. Retrieved Aug. 15, 2022.

Whitney, C., Huber, W., Klann, J., Weis, S., Krach, S., & Kircher, T. (2009). Neural correlates of narrative shifts during auditory story comprehension. NeuroImage, 47(1), 360-366. https://doi.org/10.1016/j.neuroimage.2009.04.037

Whitney, C., Kirk, M., O'Sullivan, J., Lambon Ralph, M. A., & Jefferies, E. (2011). The neural organization of semantic control: TMS evidence for a distributed network in left inferior frontal and posterior middle temporal gyms. Cerebral Cortex (New York, N.Y.: 1991), 21(5), 1066-1075. https://doi.org/10.1093/cercor/bhq180

Whitney, C., Kirk, M., O′Sullivan, J., Lambon Ralph, M. A., & Jefferies, E. (2012). Executive semantic processing is underpinned by a large-scale neural network: revealing the contribution of left prefrontal, posterior temporal, and parietal cortex to controlled retrieval and selection using TMS. Journal of Cognitive Neuroscience, 24(1), 133-147. https://doi.org/10.1162/jocn_a_00123

Zhang, S., & Li, C. R. (2012). Functional connectivity mapping of the human precuneus by resting state fMRI. Neurolmage, 59(4), 3548-3562. https://doi.org/10.1016/j .neuroimage.2011.11.023

### CONCLUSION

While the methods and systems have been described in connection with preferred embodiments and specific examples, it is not intended that the scope be limited to the particular embodiments set forth, as the embodiments herein are intended in all respects to be illustrative rather than restrictive.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; the number or type of embodiments described in the specification.

Throughout this application, various publications may be referenced. The disclosures of these publications in their entireties are hereby incorporated by reference into this application in order to more fully describe the state of the art to which the methods and systems pertain.

It will be apparent to those skilled in the art that various modifications and variations can be made without departing from the scope or spirit. Other embodiments will be apparent to those skilled in the art from consideration of the specification and practice disclosed herein. It is intended that the specification and examples be considered as exemplary only, with a true scope and spirit being indicated by the following claims.

What is claimed:

1. A method of individualized, non-invasive brain stimulation for enhanced learning comprising:
obtaining brain data of a person;
identifying one or more areas of a brain of the person for stimulation based on the brain data;
stimulating the identified one or more areas of the brain, wherein the stimulation results in enhanced learning.

2. The method of claim 1, wherein the brain data are obtained while the person is performing a learning activity.

3. The method of claim 3, wherein the learning activity is a specific learning activity such as factual learning , or skill learning.

4. The method of claim 1, wherein the brain data comprise high-resolution brain data.

5. The method of claim 4, wherein obtaining the high resolution brain data comprises obtaining high-resolution brain imaging.

6. The method of claim 5, wherein the high resolution brain imaging comprises one or more brain imaging modalities comprising magnetic resonance imaging (MRI), electroencephalogram (EEG), functional near-infrared spectroscopy (fNIRS), and magnetoencephalography (MEG) data.

7. The method of claim 6, wherein identifying one or more areas of the brain of the person for stimulation comprises joint independent component analysis (jICA) of the MRI and EEG data brain imaging modalities, or other general statistical approaches that use information from multiple brain imaging modalities to determine brain activations, such as correlation between modalities or deep learning approaches that find correspondence across modalities.

8. The method of claim 7, wherein the jICA or other analysis identifies one or more detailed MRI spatial maps and corresponding time courses, leading to an understanding of brain processes.

9. The method of claim 8, further comprising identifying one of the one or more detailed MRI spatial maps and/or corresponding time courses is most predictive of the subject's successful online learning of information.

10. The method of claim 8, wherein the identified one detailed MRI spatial map and corresponding time course is used to identify one or more greatest contributing brain areas of learning (based either on weighting value of the area through various statistical metrics, or a priori characterization of the region).

11. The method of claim 10, further comprising identifying a non-invasive brain stimulation electrode array that most closely targets the one or more greatest contributing brain areas identified from fused MRI/EEG analysis.

12. The method of claim 11, wherein identifying the non-invasive brain stimulation electrode array that most closely targets the one or more greatest contributing brain areas identified from the fused MRI/EEG analysis is done by using stimulation targeting software.

13. The method of claim 12, wherein using stimulation targeting software to identify a best fit electrode array comprises contributing an algorithm to the software that iteratively tests electric field maps (e.g. the simulated impact of stimulation from an electrode array) to find the electrode array with the closest spatial match to the jICA targets.

14. The method of claim 6, wherein the person's specific peak frequency value within each frequency band (from EEG) is used as another parameter in non-invasive brain stimulation.

15. The method of claim 1, wherein stimulation for stimulating the identified one or more areas of the brain varies over time.

16. A computer program product comprising computer-executable instructions stored on a non-transient computer-readable medium for performing a method of individualized, non-invasive brain stimulation for enhanced learning comprising:
obtaining brain data of a person;
identifying one or more areas of a brain of the person for stimulation based on the brain data; and
stimulating the identified one or more areas of the brain, wherein the stimulation results in enhanced learning.

17. The computer program product of claim 16, wherein the computer program product comprises computer-executable instructions executing on a processor, wherein the processor comprises a personal computing device such that a user of the personal computing device receives a preferred electrode array for non-invasive brain stimulation of the person after entering an identifier for the person into the personal computing device.

18. The computer program product of claim 17, wherein the personal computing device comprises a smart phone, a tablet, a laptop or notebook computer, or a personal computer.

19. The computer program product of claim 17, wherein the personal computing device interfaces with a cloud computing network.

20. The computer program product of claim 19, wherein the brain data comprise data from one or more brain imaging modalities comprising magnetic resonance imaging (MRI), electroencephalogram (EEG), functional near-infrared spectroscopy (fNIRS), and magnetoencephalography (MEG) data, and is stored in the cloud computing network and is accessible based on the entered identifier for the person.
